(19) United States
(12) Patent Application Publication
(10) Pub. No.: US 2016/0054280 A1
(43) Pub. Date: Feb. 25, 2016

(54) PUBLIC USE WATER TESTING KIOSK

(71) Applicants: PHILIP WILLIAM ROOT, BOCA RATON, FL (US); TRAVIS WILLIAM MCCAIN, LIZTON, IN (US)

(72) Inventors: PHILIP WILLIAM ROOT, BOCA RATON, FL (US); TRAVIS WILLIAM MCCAIN, LIZTON, IN (US)

(21) Appl. No.: 14/468,290

(22) Filed: Aug. 25, 2014

(51) Int. Cl. G01N 33/18; G01N 21/03

(52) U.S. Cl. CPC G01N 33/18; G01N 21/03

(57) ABSTRACT

Implementations of a public use water testing kiosk. Aspects associated with particular implementations of a water testing kiosk comprise, among others, automatically testing water based on user wants or issues, receiving a water sample for testing from the individual, providing a closeable door between the water testing and the user while the water is being tested, returning the water sample container to the user housing for the kiosk may surround all of the system parts or may be split into a housing shell and a testing unit that is inside the shell. Testing implements may be cuvette based, test strip based, probe based or other.

### CROSS-REFERENCE TO RELATED APPLICATIONS

This application is related to provisional patent applications No. 61/869,917 Titled “Self-Service Kiosk for Water Analysis”, filed Aug. 26, 2013, No. 61/869,917 Titled “Reagent Dispensing System”, filed Aug. 26, 2013, and 61/869,917 Titled “Cuvette Storage and Dispensing System” the disclosure of which is incorporated by reference and to which priority is claimed.

### REFERENCES

### US Patents

U.S. Pat. No. 1,938,544 June 1933 Schoenberg Virgil A
U.S. Pat. No. 3,240,717 September 1963 Johnson Pratt H
U.S. Pat. No. 4,640,616 December 1984 John K. Michalik
U.S. Pat. No. 4,904,605 June 1988 O'Brien et al.
U.S. Pat. No. 5,872,984 A April 1997 Berglund et al.
U.S. Pat. No. 6,395,158 November 2000 King & Millhouse
U.S. Pat. No. 6,541,269 B1 June 1994 Ramana et al.
U.S. Pat. No. 6,793,787 B1 March 2002 Hirshberg et al.
U.S. Pat. No. 8,703,057 B2 August 2006 Swanson et al.
U.S. Pat. No. 8,724,131 B1 August 2011 Gracia et al.

### Other Patents

WO2006003657 A3 July 2004 Ben David Tsur
EP19870301700 February 1986 Spani

### Websites

selmor.com.au.2013.TOSHIBA-HYCHLOR MKII DISPLAY. [ONLINE]
Available at: http://www.selmor.com.au/portfolio/toshiba-hychlor-mkii-display/.
[Accessed 1 August 13].

### BACKGROUND OF THE INVENTION

Aspects of this document relate generally to the public use kiosk for water analysis, the issuing advice, and product recommendation. A device that easily and accurately test water in a public setting, has means to offer advice for water correction, a means to recommend products, care techniques and maintain records, which provides advantages to many.

Water testing devices for aquatic samples removed from the original body of water are commonly used to solve problems and maintain bodies of water. Conventionally when an individual cares for a body of water, one seeks advice to properly maintain water quality and/or to correct issues observed, typically performed with a series of water test(s).

In retail setting, a store's employee typically uses colorimetry, refractometry and/or probe(s), on a provided sample from the care taker where the water originates from aquatic samples, removed from the original body of water such as aquariums, ponds, tanks, lakes, rivers, streams, pools, wastewater, runoff, or any other type of water to test water and make a determination. The use of certain prior art products such as U.S. Pat. No. 6,395,158 by King & Millhouse, U.S. Pat. No. 6,793,787 by Hirshberg et al., U.S. Pat. No. 4,640,616 by Michalik U.S. Pat. No. 1,938,544, give results in unit of measure. Although the units of measure are desired; a unit of measure along with a corrective course of action for an individualized situation is more desirable.

Upon completion of said test, results are communicated to customer with advice and/or methods to correct any issue(s) that can or may persist within the original body of water. One skilled in the area of water testing understands that these methods are inadequate at times as different employees recommend different actions and/or brands of products to correct the same problem(s). Employees must be trained on how to use these devices and on ways to correct problem(s). Records are not typically kept for reference. In situations where records are kept said records are not readily accessible to the customer. Known within the community said testing and methodology is greatly errored as: (1) methodology varies from community to community, retail store to retail store, employer to employer, employee to employee and care taker to care taker, (2) said type and/or methods of water testing analysis are affected by high rates of human error, (3) filing/record keeping of said testing and results not being provided or not cost effective.

The most common method(s) of water testing determination uses colorimetric analysis, which utilizes colorimetric reagents to ascertain concentration levels of chemical elements and compounds, Pratt's U.S. Pat. No. 3,240,717 and colorimetric testing strips, O'Brian et al. U.S. Pat. No. 4,904,605, with comparable charts of visible color change. Said methods require individualized involvement and assessment; by adding water to colorimetric reagents strips and analyzing results with comparison charts. Said methods' shortcomings parallel the said errors found in paragraph [0011].

Colorimeter, Schoenberg U.S. Pat. No. 1,938,544, device(s) measures the absorbance or transmission of particular wavelengths of light by a specific solution. Colorimeters are used to determine the concentration of a known solute in a given solution by the application of the Beer-Lambert law. With the current platform in the market, colorimeter testing requires an individual to place a water sample into a provided cuvette. The cuvette is placed in the colorimeter for a base line measurement and then taken out of the colorimeter. The said sample, within the cuvette is then mixed with proper reagent(s) solutes. After reagent(s) have reacted with the solute the said sample is then placed back into the colorimeter and read in either absorbency or in an appropriate unit value, which is then communicated to the caretaker. Said testing technology is the most accurate, yet concerns surrounding human error, (1) device reading fingerprints left on cuvettes (2) cuvette wrongly loaded (3) lack of knowledge on said device (4) and improper execution time of test often leads to in-accurate test results.

A refractometer, provides analysis of salt levels in different bodies of water. An individual uses either an analog device or an electromechanical device/digital reading of the refractive index, Michalik U.S. Pat. No. 4,640,616. The limiting factors for said devices (1) only give the refractive index an estimated salt content, (2) do not differentiate between the varieties of salts (3) currently parameters can not be tested by refractometry, (4) proper cleaning and calibration must be performed on such devices.

Probes, King & Millhouse U.S. Pat. No. 6,395,158 and Hirshberg et al. U.S. Pat. No. 6,793,787, are generally placed within a sample of water for a digital reading. Probes limiting factors include (1) calibration, (2) affected by other untargeted elements (3) depending on the environment, samples may need to be electrically isolated, (4) difficult to maintain, (5) cross contamination issues when testing multiple water samples.

Selmor Displays, an Australian company has a pool water testing system (http://www.selmor.com.au/portfolio/toshiba-hychlor-mkii-display/). This system is used by the user to test the pH levels of their pool. The user would follow the prompts on the touch screen then print a shopping list with the products needed to fix their pool. This system is only an informational system and it dose not test water. So the need for a person to make a subjective interpretation of color still exist but the training in regards to product recommendation and knowledge of water chemistry is overcome. This system does not store users records or make records accessible to users over the web.

Currently very few, if any, records are kept of customers' test results, as record keeping is cost prohibitive and a timely endeavor, under the current method(s), in a retail environment. An automated record keeping system that could quickly analyze historical data, would improve time, cost and advice.

An information system like Selmor's combined with ability of testing water with a known testing method like the ones in paragraphs [0018], [0019], [0021] would be of great value to many industries.

A water testing device that overcomes problems in paragraphs [0016]-[0023] associated with prior art would be valuable to many trades.

### BRIEF SUMMARY OF THE INVENTION

Aspects associated with particular implementations of the public use water testing kiosk are: a testing implement, control system, sensors, a closable door between user and mechanisms, a system for providing information to the user regarding a course of action, housing, printer, waste receptacle, a graphical user interface, a payment system, and a receiver for a water sample in the housing that is accessible to a user from the outside of the housing. It should be noted, not all implementations require every aspect of listed implementation.

An individual would use the kiosk's graphical user interface to either choose desired test or select a current problem(s) and body of water type, which would allow the system to suggest testing. Once test selection is complete the user would place a water sample to be tested in the water sample receiver. The testing implement would analyze the water, and results would be provided.

In the first particular implementation of the public use water testing kiosk, the kiosk is comprised of a housing, a water sample receiver accessible to an individual outside of the housing, a trash receptacle for used consumables, a waste water reservoir for spilled or discarded water, a graphical user interface coupled to the housing and responsive to the controller, said graphical user interface configured to prompt a user to submit water type, identify issues and supply information regarding which test to perform, and a printer.

In the second particular implementation, of the public use water testing kiosk, the kiosk is comprised of a housing, a water sample container receiver, accessible to an individual, outside of the housing, a trash receptacle for used consumables, a waste water reservoir for spilled or discarded water, graphical user interface coupled to the housing and responsive to the controller, the graphical user interface configured to prompt a user to submit water type, identify issues and supply information regarding test to perform, a printer, a motorized rotor with multiple reagent storage systems preferably move said reagent systems to cuvettes, a fixture removing water from the sample container, a fixture removing reagent from reagent storage, a light source and optical sensor placed for light to travel through the cuvette, preferably in cuvette rotor, an optical system for measuring liquids dispensed from the reagent systems, a cuvette storage and dispensing system, a system that moves a cuvette from storage system into a cuvette rotor, a cuvette rotor with multiple slots for cuvettes, so that the cuvettes are moved in a circular path to preferred locations and preferably able to move back and forth motion, cuvettes to mix reagents and a system for discarding cuvettes upon completion.

In the third particular implementation, of the public use water testing kiosk, the kiosk is comprised of a housing, a water sample receiver accessible to an individual outside of the housing, a trash receptacle for used consumables, a waste water reservoir for spilled or discarded water, a graphical user interface coupled to the housing and responsive to the controller, the graphical user interface configured to prompt a user to submit the water type, identify issues and supply information regarding test to perform, a printer, a fixture removing water from sample container, a storage system of reagent strips, a test strip shuttle, a light source, and optical sensor for detecting color.

In the fourth particular implementation, of the public use water testing kiosk, the kiosk is comprised of a housing, a water sample receiver accessible to an individual outside of the housing, a trash receptacle for used consumables, a waste water reservoir for spilled or discarded water, a graphical user interface coupled to the housing and responsive to the controller, a graphical user interface configured to prompt a user to submit the water type, identify issues and supply information regarding test to perform, a printer, a set of probes to analyze water.

Water testing method, for a public use water testing kiosk, consists of receiving water from a user, testing water sample, returns the water sample container to the user, upon completion, displays results on the graphical user interface, and issuing an optional printed report to the user.

Water testing method water at a public use water testing kiosk, consists of interacting with a graphical user interface, receiving the water sample from the user, and return the water sample container to the user upon completion, displaying results on the graphical user interface and issuing an optional printed report to the user. The kiosk housing encloses water testing device such that the user cannot access test equipment from outside the housing while user interacts with the kiosk. The sample water is dispensed into at least one cuvette(s) and an optical measurement of the water is taken, preferably one or more reagent(s) are added to cuvette containing sample water, the reagent(s) and sample water are mixed by the cuvette rotor motion, a second optical measurement is taken, of said water sample, and the cuvette is then discarded.

Water testing method at a public use water testing kiosk, consists of interacting with a graphical user interface, user taking a test strip from a holder on outside of kiosk, dipping strip in customer provided water sample, user then places activated test strip into test strip receiver outside of housing, displaying the results on the graphical user interface, and issuing an optional printed report to the individual.

Water testing method at public use water testing kiosk, consists of a user placing sample water in receiver, automatically making contact between test strips and users water, and a test strip reader reading said test strip, and the said test strip is then discarded, displaying the results on the graphical user interface, and issuing an optional printed report to the individual.

Water testing method at a public use water testing kiosk, comprises of placing sample water in receiver, an automatic reel of test automatically making contact between test strips and users water, and a test reel reader reading said test strip, the said test strip is then discarded, displaying the results on the graphical user interface, and issuing an optional printed report to the individual.

Water testing method at a public use water testing kiosk, comprises of placing sample water in receiver, a set of probes making contact with users water, displaying the results on the graphical user interface, and issuing an optional printed report to the individual.

A cartridge for dispensing cuvettes, that contains an opening for cuvettes to exit, is tape fed, does not require the movement of all the cuvettes in the cartridge upon the advance of a single cuvette, is powered by an outside source, and does not contain ribs or channels.

A method generating water care recommendations on information gathered from a public use water testing kiosk, the method consists of using comprising of stored data related to the current test results, retrieving individual's previous data, analyzing data from the current test results, analyzing data from any previous results, issuing a printout containing advice.

The objectives of the present invention are to provide:

A means to easily and accurately test water,

A means to offer advice for correction of water problems,

A means to recommend products and care techniques,

A means to have records of results and advice,

This public use water testing kiosk makes a determination of what parameters to test based on issues selected from a list of common problems displayed, test water, suggest products and methods to correct issues provides advantages through consistent advice, reduction of employee training and maintenance, and manpower not being required to assist a user.

The foregoing and other aspects, features and advantages will be apparent to those artisans of ordinary skill in the art from the DESCRIPTION and DRAWINGS and from the CLAIMS.

### BRIEF DESCRIPTION OF THE DRAWING

Particular illustrative implementations will hereinafter be described in conjunction with the appended drawings, where like designations denote like elements, and:

FIG. 1 is a front perspective view of the preferred embodiment of public use water testing kiosk with all access panels closed illustrating the components that make up the preferred embodiments and their related elements.

FIG. 2 is a back view of a preferred embodiment of public use water testing kiosk for water testing with access panels closed.

FIG. 3 is a front perspective view of the public use kiosk for water testing with the access panels open illustrating the components that make up the preferred embodiments and their related elements.

FIG. 4 is a back perspective view of the public use kiosk for water testing with the access panels open illustrating the components that make up the preferred embodiments and their related elements.

FIG. 5 is a front perspective view of the public use kiosk for water testing with the front access panel removed illustrating the components that make up the preferred embodiments and their related elements.

FIG. 6A is a front perspective view of a preferred embodiment of a cuvette based water testing system for a public use kiosk for water testing.

FIG. 6B is a back view of a preferred embodiment of a cuvette based water testing system for a public use kiosk for water testing.

FIG. 7A is a perspective view of a preferred embodiment of a reagent extractor removed from testing implement.

FIG. 7B is an exploded view of FIG. 7A.

FIG. 8A is a perspective view of a preferred embodiment of a user water extractor removed from testing implement.

FIG. 8B is an exploded view of FIG. 8A.

FIG. 9A is a bottom perspective view of the preferred embodiment of the slide plate assembly used in a cuvette based water testing system for a public use kiosk for water testing.

FIG. 9B is an exploded, top perspective view of the preferred embodiment of the slide plate assembly used in a cuvette based water testing system for a public use kiosk for water testing.

FIG. 10 is a bottom perspective view of the preferred embodiment of the cuvette rotor used in a cuvette based water testing system for a public use kiosk for water testing.

FIG. 11 is a perspective view with the top cover removed of the preferred embodiment of the internal workings used in a cuvette based water testing system for a public use kiosk for water testing.

FIG. 12A is a perspective view of the preferred embodiment of the water sample container receiver.

FIG. 12B is an exploded view of FIG. 12A.

FIG. 13A is a side view of the preferred embodiment of the cuvette loader used in a cuvette based water testing system for a public use kiosk for water testing.

FIG. 13B is a perspective view of the preferred embodiment of the cuvette loader used in a cuvette based water testing system for a public use kiosk for water testing.

FIG. 13C is an exploded view of the preferred embodiment of the cuvette loader used in a cuvette based water testing system for a public use kiosk for water testing.

FIG. 14A is a perspective view of a cuvette used in a cuvette testing implement.

FIG. 14B is a left view of a cuvette used in a cuvette testing implement.

FIG. 14C is a back view of a cuvette used in a cuvette testing implement.

FIG. 14D is a top view of a cuvette used in a cuvette testing implement.

FIG. 14E is a front view of a cuvette used in a cuvette testing implement.

FIG. 14F is a right view of a cuvette used in a cuvette testing implement.

FIG. 15A is a back perspective view of the preferred embodiment of a reagent storage system.

FIG. 15B is a front perspective view of the preferred embodiment of a reagent storage system.

FIG. 15C is a cut away view of the preferred embodiment of a reagent storage system tip.

FIG. 16A is a top perspective view of the preferred embodiment of a cuvette dispensing and storage system.

FIG. 16B is a top perspective view of the preferred embodiment of a full cuvette dispensing and storage system with the top removed.

FIG. 16C is exploded view of the preferred embodiment of a cuvette dispensing and storage system empty.

FIG. 16D is a cut away view of the preferred embodiment of a cuvette dispensing and storage system at the point of exit.

FIG. 16E is a cut away view of the preferred embodiment of a cuvette dispensing and storage system at the gear mesh.

### DETAILED DESCRIPTION OF THE INVENTION

This disclosure, its aspects and implementations, are not limited to the specific components or assembly procedures disclosed herein. Many additional components and assembly procedures known in the art consistent with the intended water testing kiosks and/or assembly procedures for water testing kiosks will become apparent from this disclosure. Accordingly, for example, although particular water testing kiosks are disclosed, such systems and implementing components may comprise any shape, size, style, type, model, version, measurement, concentration, material, quantity, and/or the like as is known in the art for such systems and implementing components, consistent with the intended operation of water testing kiosks.

Water testing kiosks specifically described in this disclosure and which will become apparent from the explanation provided in this disclosure may include one or more of the various aspects relating to water testing kiosks discussed herein. The various aspects may be taken together or separately for various combinations and sub-combinations of aspects and system components to assemble a water testing kiosk having any number of configurations depending upon the ultimate use of the system, features included and cost of the system desired. Those of ordinary skill in the art will readily be able to assemble a system once the principles discussed and combinations explained are understood.

As used herein, the term “testing kiosk” refers to the type of testing kiosk wherein a user does not have access to the testing system and, therefore, can safely use the kiosk without risk of coming in contact with chemicals or effect the results of the test by improper handing of testing equipment.

A first aspect of a testing kiosk relates to an overall system layout. Although particular configurations may be shown in the related Figures, other configurations are also contemplated and described throughout this disclosure. Each of the configurations described here includes: a user interface and a water testing implement. Additional optional components included in these particular configurations include: a payment receiver, printer, cellular card, barcode reader, WIFI antenna, and other optional elements.

A control system for a water testing kiosk may be configured with software and/or hardware configured to provide self diagnostics for the system as well as reporting modes that allow for direct wired or wireless reporting to a central computer through the Internet or other appropriately configured local area or wide area network (LAN or WAN). Alternatively, reporting and/or data collection may be done by any other method known in the art for sales and inventory tracking.

Whether through an existing internal connection or through a periodic connection through an external computer connection such a USB port, particular implementations of a water testing kiosk may comprise an external connection to allow memory stick upgrade of the system controls and touch screen interface by store manager when new testing consumables are added or other software or system updates are desired. Automatic updating through the existing, internal connection, if included, is also contemplated. Other particular implementations may also comprise external video connections through which a point of purchase display may be regularly updated and show still and/or moving images for marketing. Yet other particular implementations may comprise a security camera to monitor system use and abuse, and store and/or send those images to the system owner or store management.

A water testing kiosk may also be configured to include training for the user and/or for the technician/store worker. For example, a video explanation of how the system works, for marketing and/or for step-by-step explanation while the user is testing water may be programmed into the control system and user interface. Additional tutorials and explanations may be programmed in for a service technician, a store manager or other person interfacing with the system to simplify its use. For example, an explanation of how to run diagnostics for the system, how to change consumables, and/or how to swap out modular level components like the touch screen interface, testing system, and marketing display.

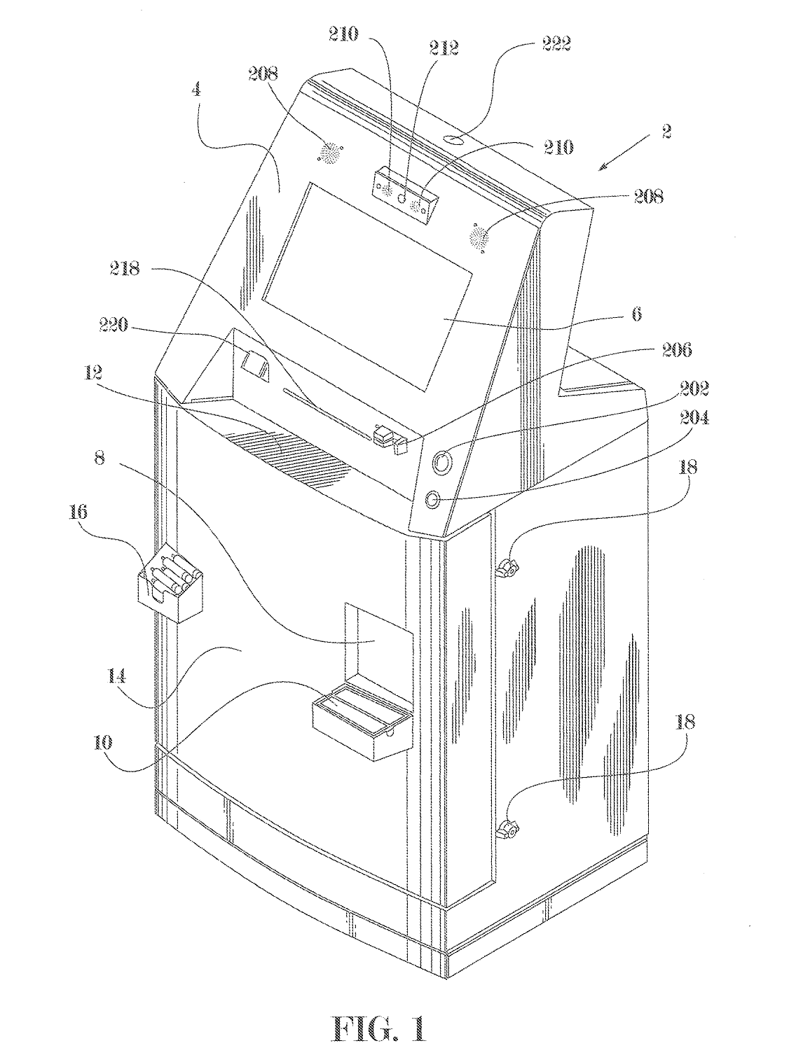

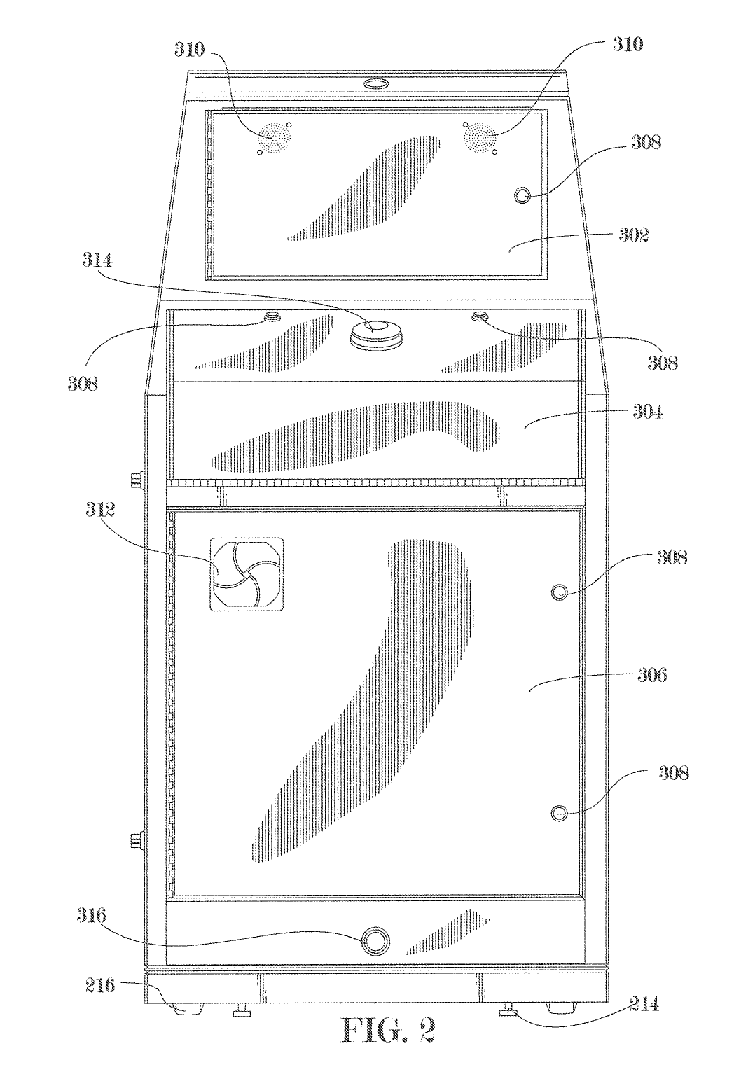

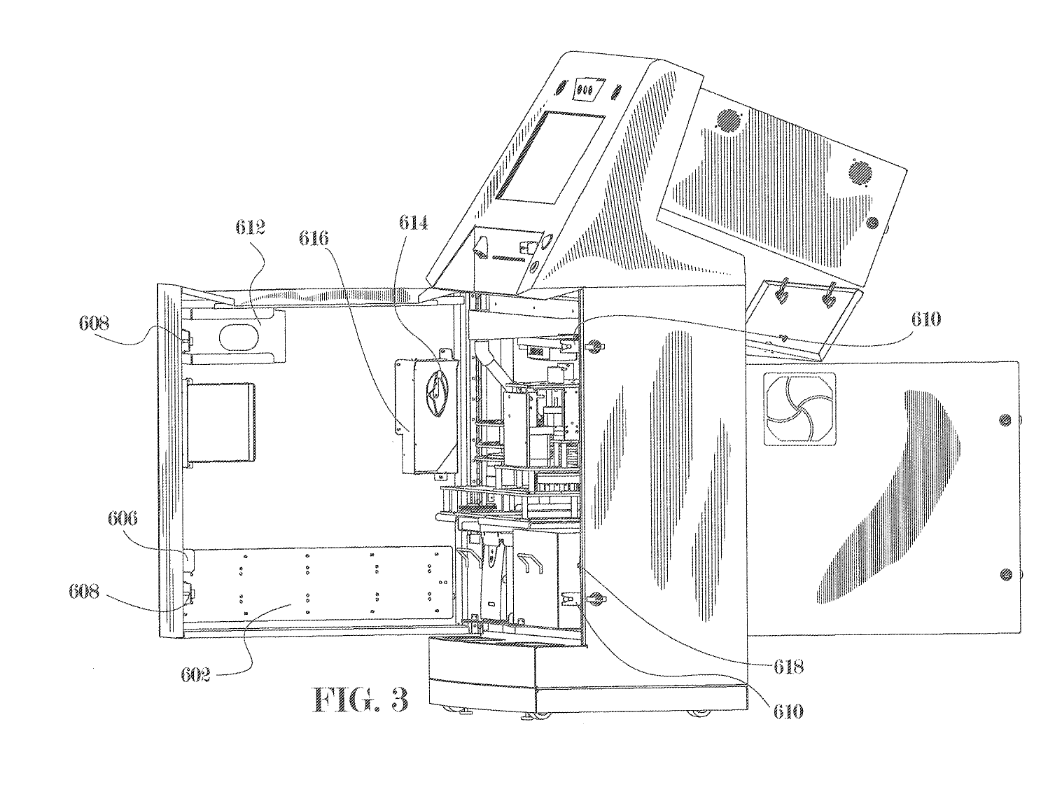

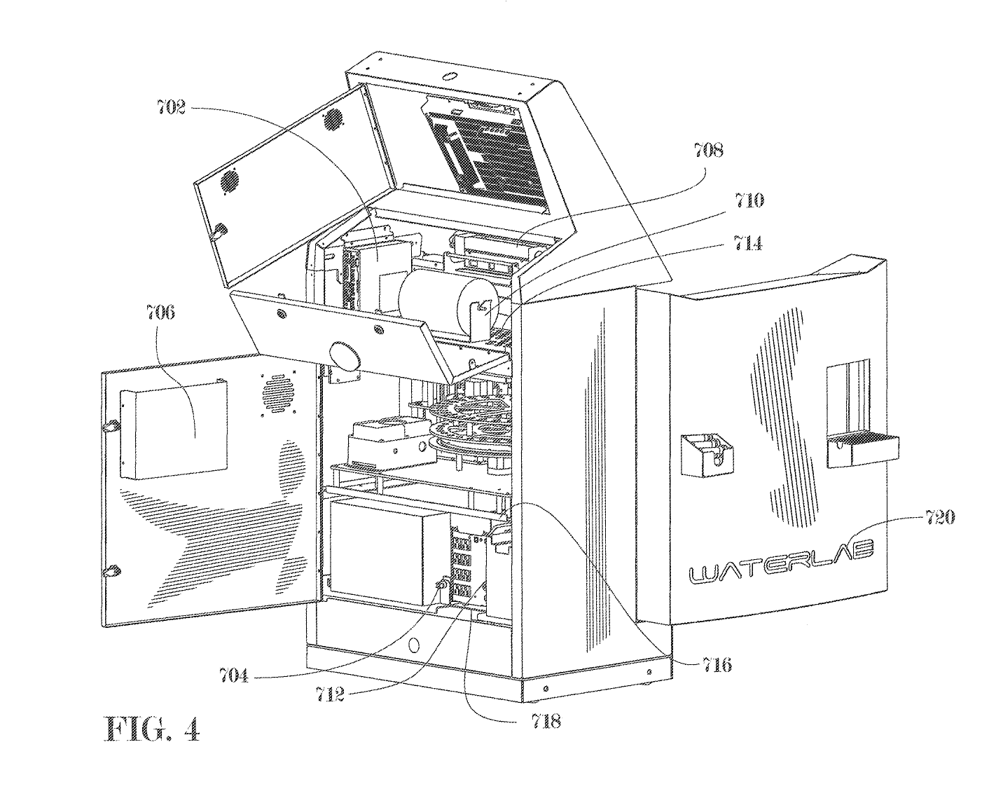

FIG. 1 illustrates front perspective view of a water testing kiosk specifically configured according to a particular implementation as a testing kiosk. The testing kiosk 2 comprises a housing shell 4 comprising of a touch screen display 6 is operatively associated with a system controller see FIG. 4,702 located, in this particular implementation, in the housing shell 4. The housing shell 4 comprises a access door 14 with a water sample receiver opening 8 with a drip plate 10, a sloped water sample preparation area 12, a holder for empty water sample containers see FIG. 3, 616, a dispenser said empty water sample containers 16 and a set of locks 18 with alignment pins see FIG. 3, 608. The housing shell 6 comprises of 3 openings see FIG. 2, 302, 304, 306 in its rear side which may or may not be enclosed with a door or panel. In this embodiment opening 302, 304, 306 has locks 308 to secure systems from the general public. The main controller may be configured to stop operation of the testing implement unless the door 16 is closed. A assess door switch see FIG. 3, 618 and switch depressor FIG. 3, 606 monitors this connection. The system may also have similar switches for deactivating the system 704 and 712 (See FIG. 4), for waste water reservoir see FIG. 5, 904 and waste bin 908. These switches may be used to also indicate whether it is possible that maintenance tasks were completed. When a device is removed the switch is deactivated and a signal is sent to the controller see FIG. 4, 702 A keyboard is located in slot see FIG. 5, 902 for maintenance task and accessing controller.

Sloped water sample preparation area see FIG. 1, 12 is a place for a user to place water into a water sample container if they did not arrive at the water testing kiosk in a expectable sample container or has excess water that needs to be discarded. This is pitched to slope away from the user to channel any spilled or unneeded water. This funnels water in to a trey see FIG. 5, 912 that has piping 910 to move water to waste water reservoir 904. Waste water reservoir 904 is equipped with a water level sensor that interacts with the controller to alert staff of conditions.

Fans see FIG. 2, 310 blow air out while fan 312 filters air before air is input into housing shell 4, to remove any heat that may occur from electronic in the housing shell. Shelf see FIG. 4, 714 is perforated to help with air flow inside of unit.

Shelves 714, 716, 718 are included in this embodiment to allow for easy removal of components. Other embodiments may have kiosk assembled as a single unit.

In FIG. 1 the housing shell 4 comprises of a card reader 206 and barcode reader 220 these components could be used for accepting payment, identifying user, loyalty programs, reports or entering data into a system. Information or instructions would preferably be issued through touch screen and printer slot 218. A printer see FIG. 4, 708 and paper holder 710 enclosed in the housing shell 4 issues results and recommendations.

FIGS. 1 and 2 also shows a possible location for some of the additional communication devices such as speakers 208, microphone(s) 210, WIFI antenna 314 and a camera 212. These devices could be used to provide remote trouble shooting with users, hardware and make updates easier. It is contemplated that a live operator assists users with these devices when software is not robust enough to solve the problem or issues are not corrected after many attempts.

It may also be desired to have casters 216 on units for the convenience of moving the kiosk. Casters may need a system for braking such as braking system 214. This is to secure system from movement while a user is performing test. The braking system demonstrated here is a set of levers with access located inside a compartment see FIG. 5, 802 under the main access door 14.

FIG. 1 also shows a possible way of making a water testing kiosk American with Disability Act compliant with the use of a roller ball 202 and a push button 204.

Power and/or network cables can enter through opening see FIG. 2, 316. It is preferred to have all electronics run through an uninterruptable power supply see FIG. 5, 906 like U.S. Pat. No. 5,872,984A this feature is used to account for the unknowns of power conditions in many different locations.

FIG. 3 displays preferred safety equipment including gloves 612, safety glasses 614, and operating instruction bin see FIG. 4, 706. Other safety equipment may be stored under waste bin see FIG. 5, 908 under shelf 718.

A set of LED's mounted in panel FIG. 3, 602 are used to light a logo on the front of the unit see FIG. 4, 720. These LED's can indicate maintenance needs to employees without distracting customer by changing colors.

FIG. 1 shows an opening 222 on the top of this embodiment. This opening may be used for additional displays. Such displays maybe used as a means of advertising the kiosk or other desired products. A tube could raise an additional monitor off the top of the housing shell 4 and house the signal wire and power.

A user will interact with a graphical user interface see FIG. 1, 6 to determine test routine for the user to preform test. User may select either a predefined bundle of test, use a trouble shouter to select test, or choose parameter individually.

The water sample container receiver see FIG. 12A, 2100 protrudes from housing through shell feature see FIG. 1, 8 when opened on a shaft see FIG. 12A, 2102 that is locked in place by the water sample receiver top see FIG. 12B, 2126 and the water sample receiver bottom see FIG. 12B, 2124 interacting with features see FIG. 12B, 2132 and 2134 it is nested and secured when assembled with panel 2120 and cover to match out side of kiosk 2106. When a user places a sample container see FIG. 12B, 2112 into the water sample receiver 2100 a sensor FIG. 12B, 2116 detects that a sample container is inserted. A block FIG. 12B, 2118 holds the bottom of the users sample container and shields any water from sensor FIG. 12A, 2110. The user is prompted to close the sample container receiver 2100. It is contemplated that this be closed by a mechanized device. The controller recognizes the door is closed by feature FIG. 12A, 2101 passing through an optical interrupter see FIG. 11, 2204. The door is held closed by a electromagnet see FIG. 11, 2202 interacting with a bar of metal 2102 on the door. A spring FIG. 12B, 2122 keeps pressure on the door so as when the electromagnet is powered off the door automatically opens. The part feature FIG. 12A, 2114 acts to catch the door from opening too far and hitting the housing shell 4.

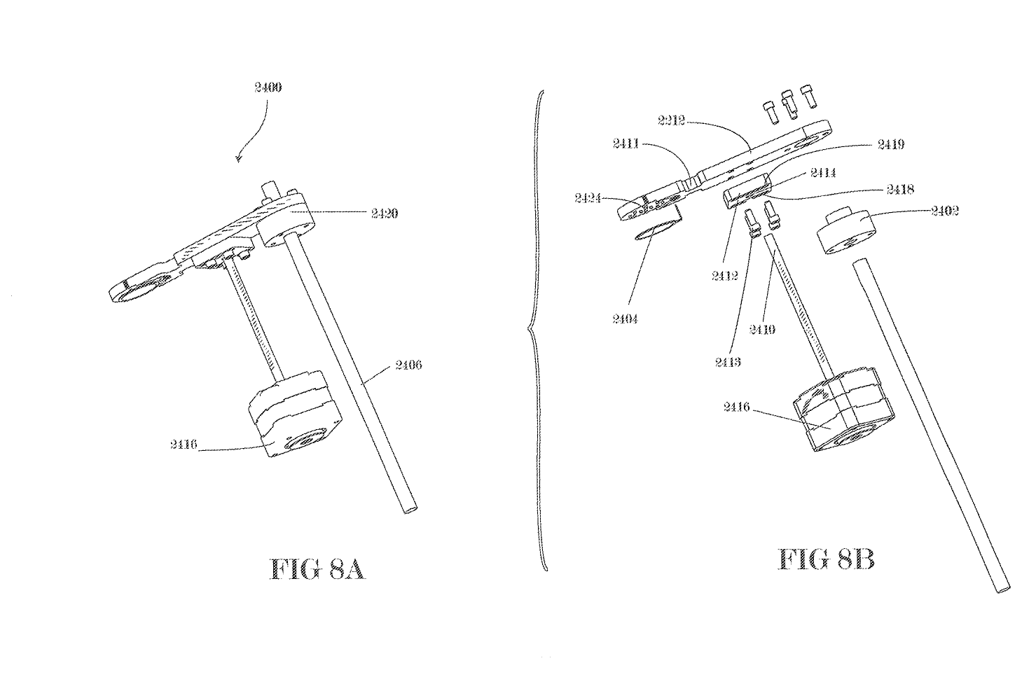

Once the door is held in place by the electromagnet FIG. 11,2202 the amount of water in the container is checked. This is checked by a linear motor FIG. 11, 2416 attached to plate see FIGS. 8A and 8B, 2212 applies rotational force to the linear screw 2410, the screw transfers the motion to plate 2412. Linear screw 2410 sets in feature 2411 and is attached to plate 2412 with a set screw. Plate 2412 is attached to depressor 2212 through 2 linear slots 2418, 2419 these slots have screws 2413 that allow for movement front to back to correct any alignment issues with liner screw 2410 and liner slide 2406 not being parallel. The linear stepper moves down the liner slide 2406 on liner bearing 2402 until the touch sensor 2404 makes contact with the water sample container. Sharp edged bumps 2424 on depressor plate 2422 allows this sensor to be more sensitive.

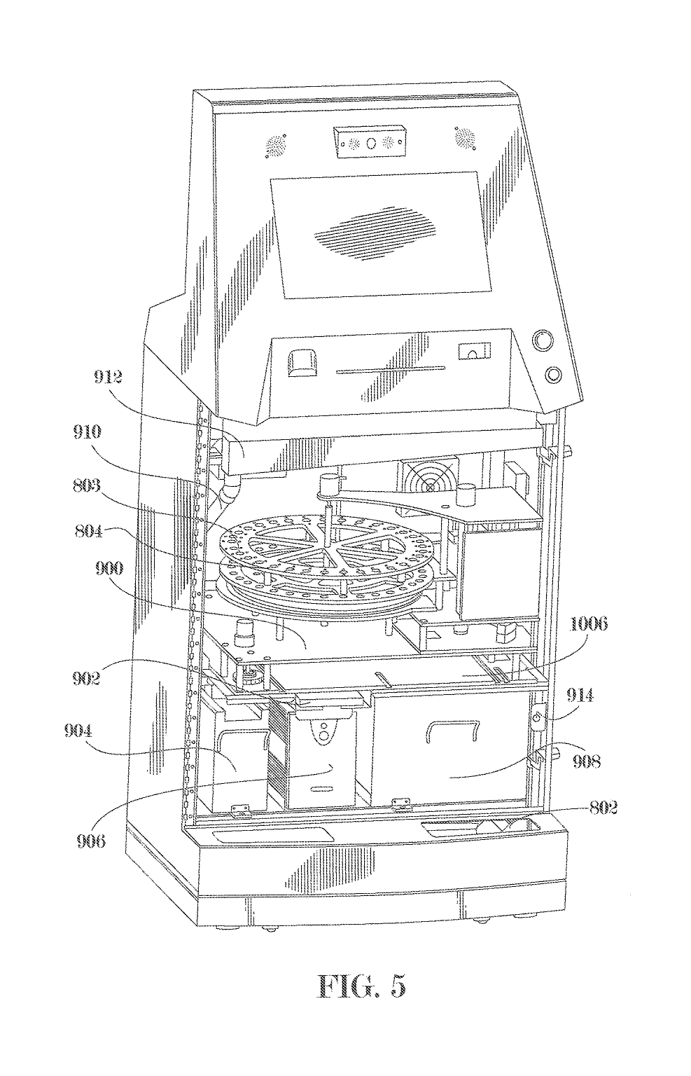

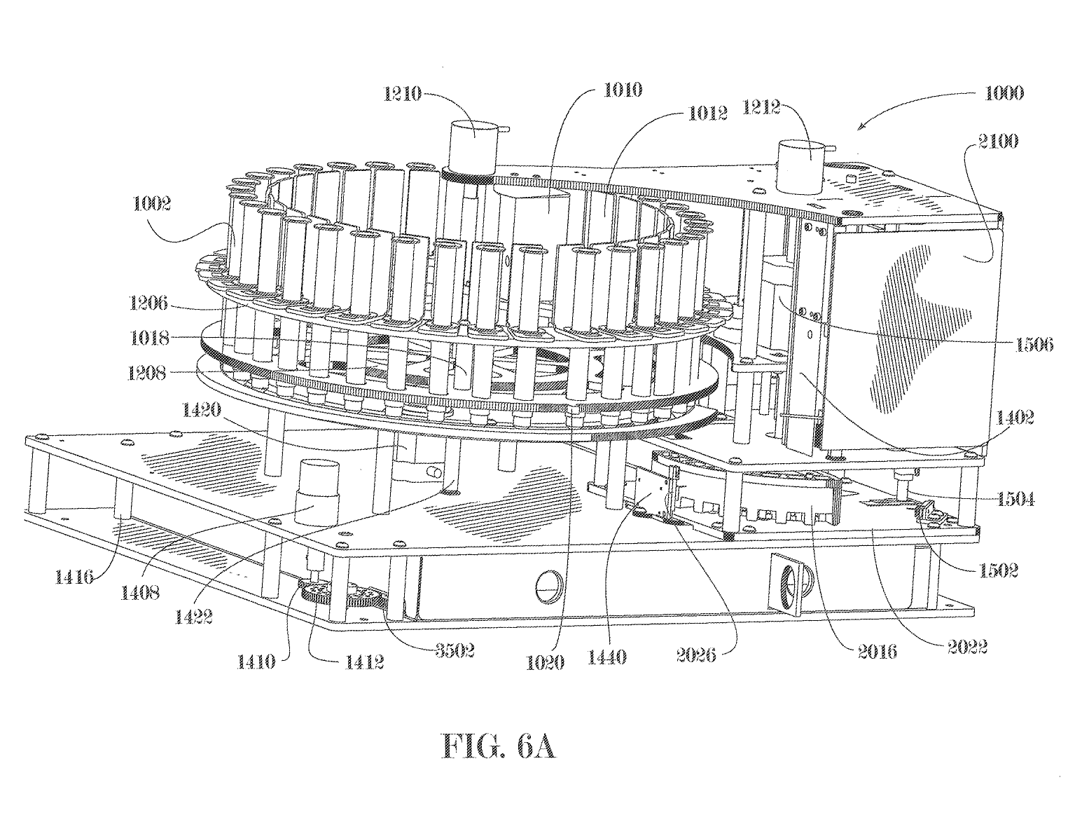

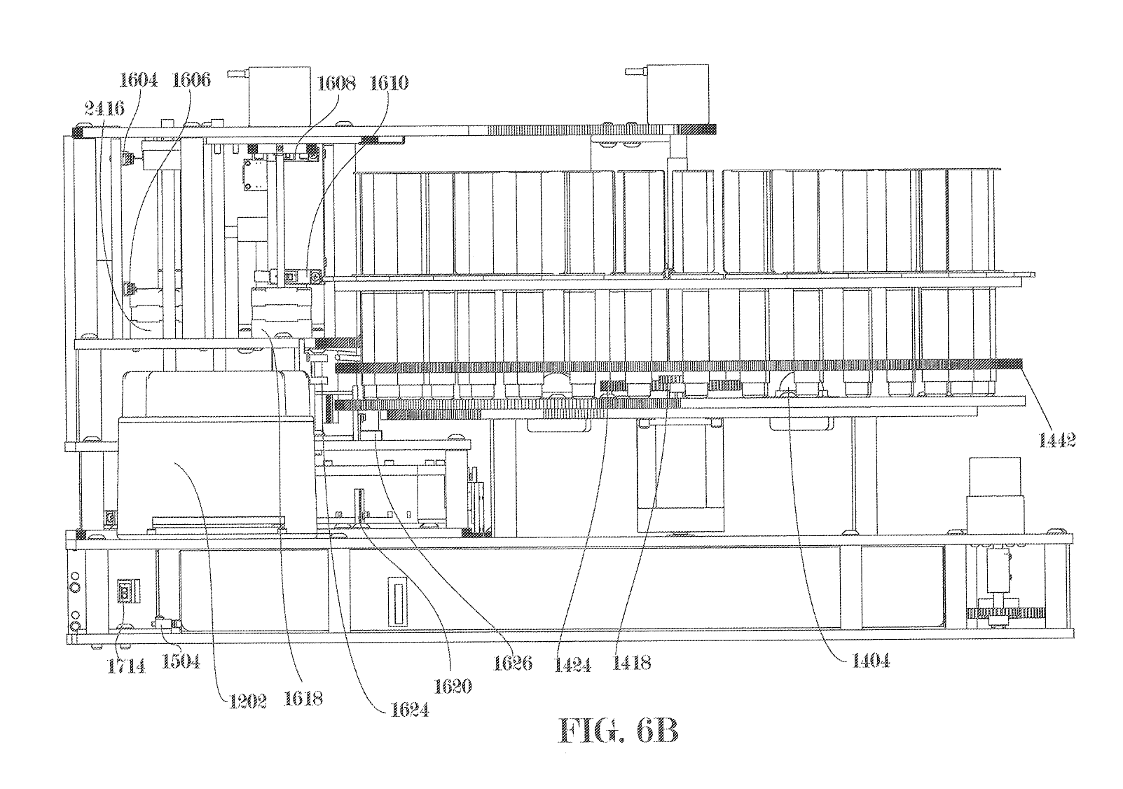

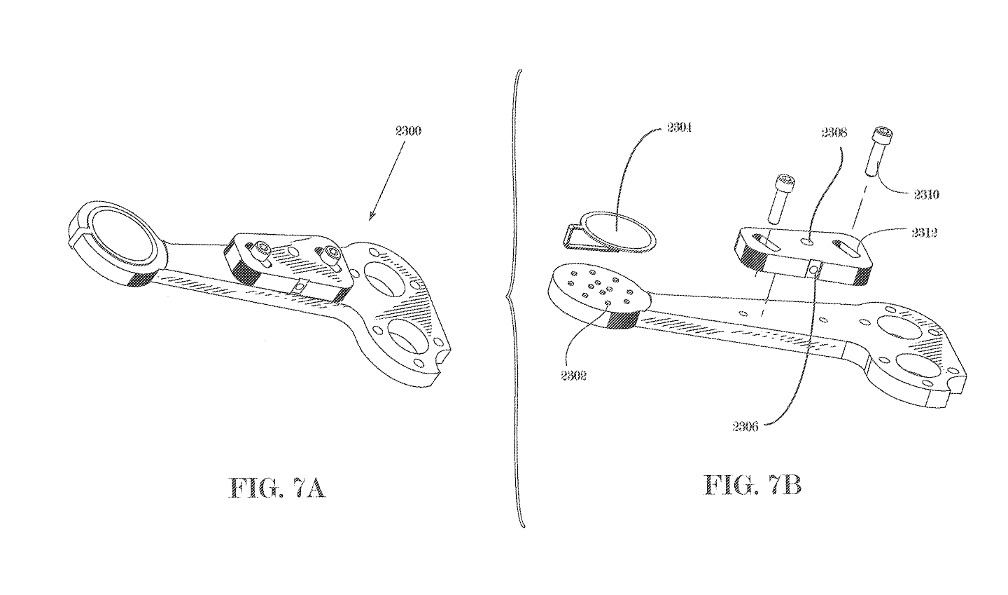

This depressor protrudes through panel see FIG. 11, 1102 through slot 2206, that does not allow the user to see or place objects other than sample into the kiosk. The height of depressor at contact time is passed to the controller for a calculation to determine if enough water is present to run requested test. The height is calculated by the number of steps the motor has taken from one of the sensors FIG. 6B,1604 and 1606 at its resting points. If it has been determined that enough water is present testing may begin else the user will be told to add more water or modify selection.

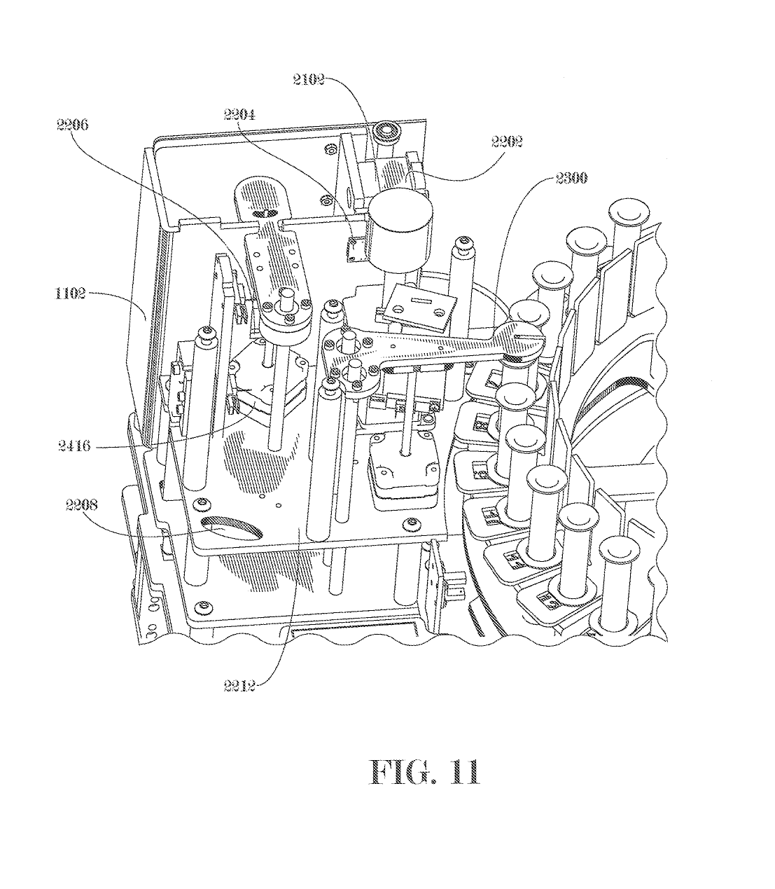

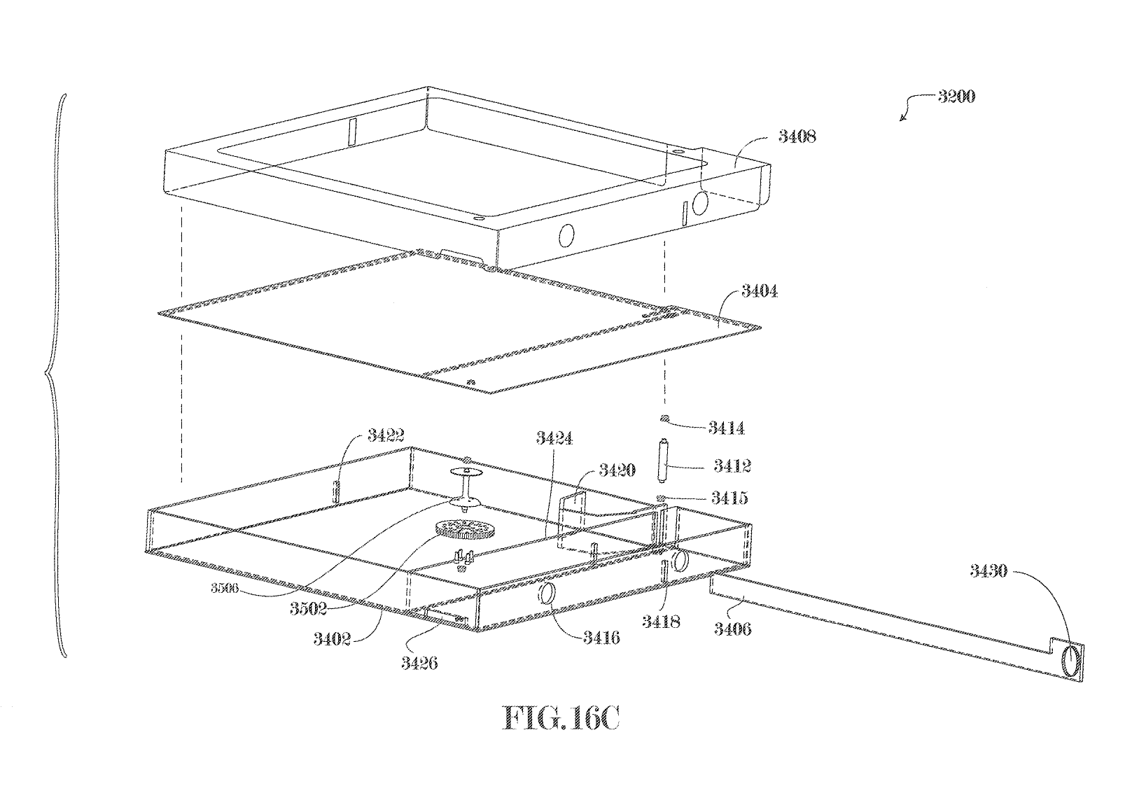

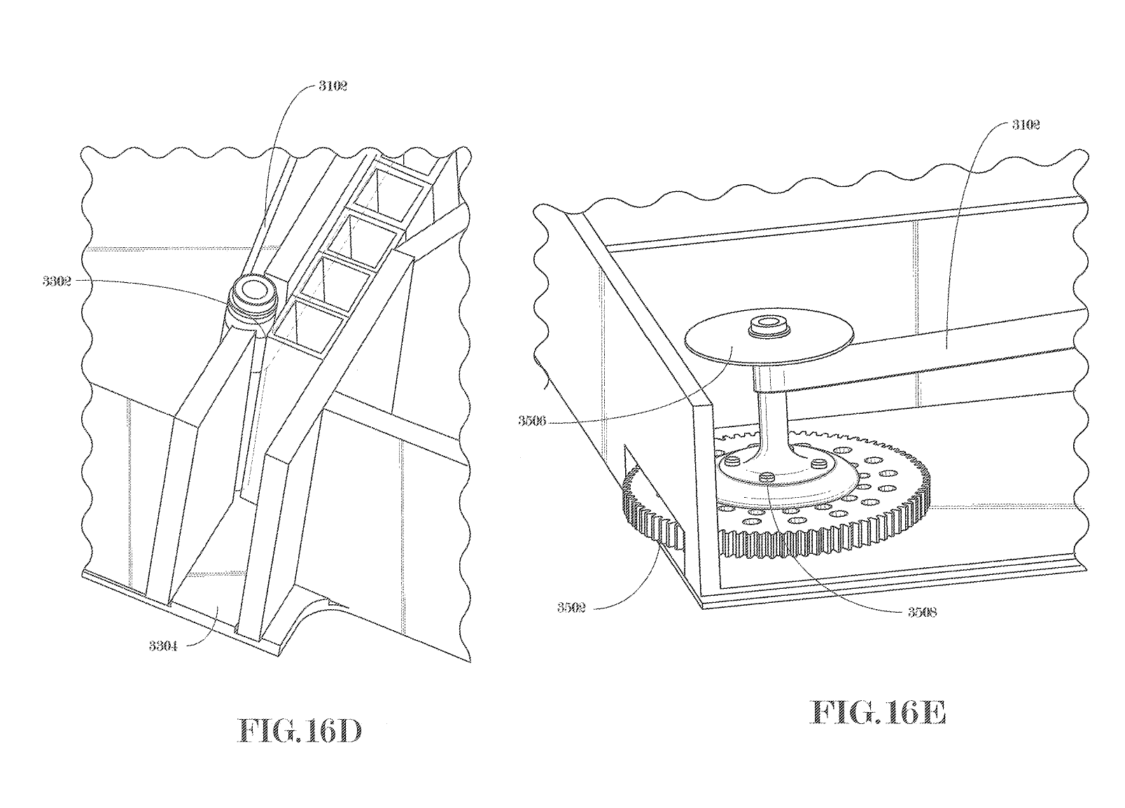

FIG. 6A is a view of a water testing implement specifically configured according to a particular implementation as a testing kiosk that uses cuvettes to test water. This system will sit inside an embodiment of the housing shell 4 of the testing kiosk. After water has been added to kiosk in an embodiment where the cuvette system 1000 is used, a linear actuator 1504 is moved to the down position until face see FIG. 13C, 1720 is below surface see FIG. 16D, 3304. This moves lifter pin see FIG. 13C,1718 down. If another cuvette is obstructing the way the chafer feature see FIG. 13A 1702 clears the obstruction by pushing the cuvette back into the cuvette cartridge FIG. 16A,3200. This will cycle several times until face see FIG. 13C, 1720 moves the cuvette back in to the cuvette cartridge and is able to move below surface see FIG. 16D, 3304.

Motor see FIG. 6A, 1408 is used to drive a gear 1410 that transfers energy to gear 1412 to pass motion to gear 3502 inside cuvette cartridge see FIG. 16C, 3200.

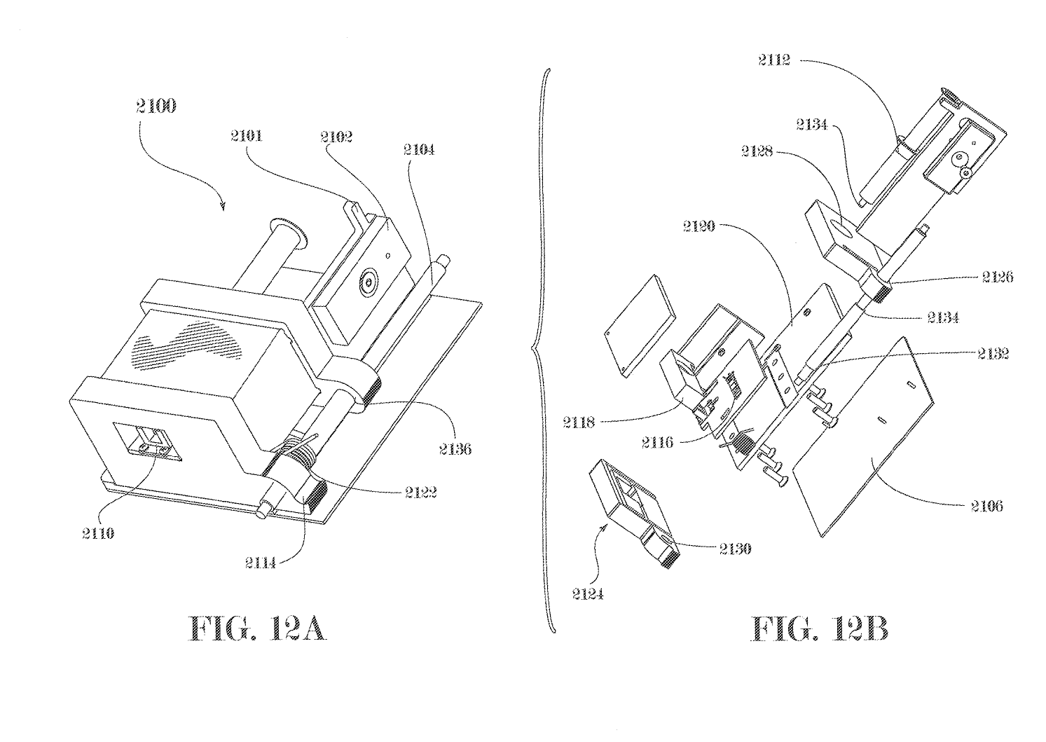

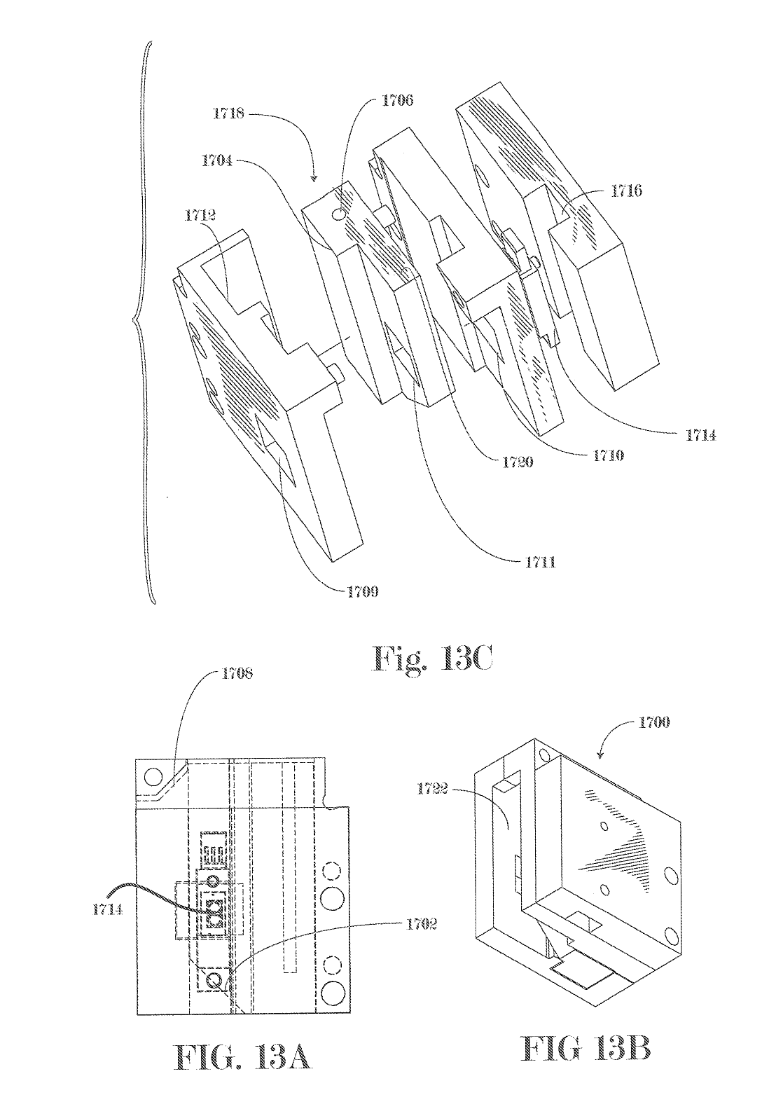

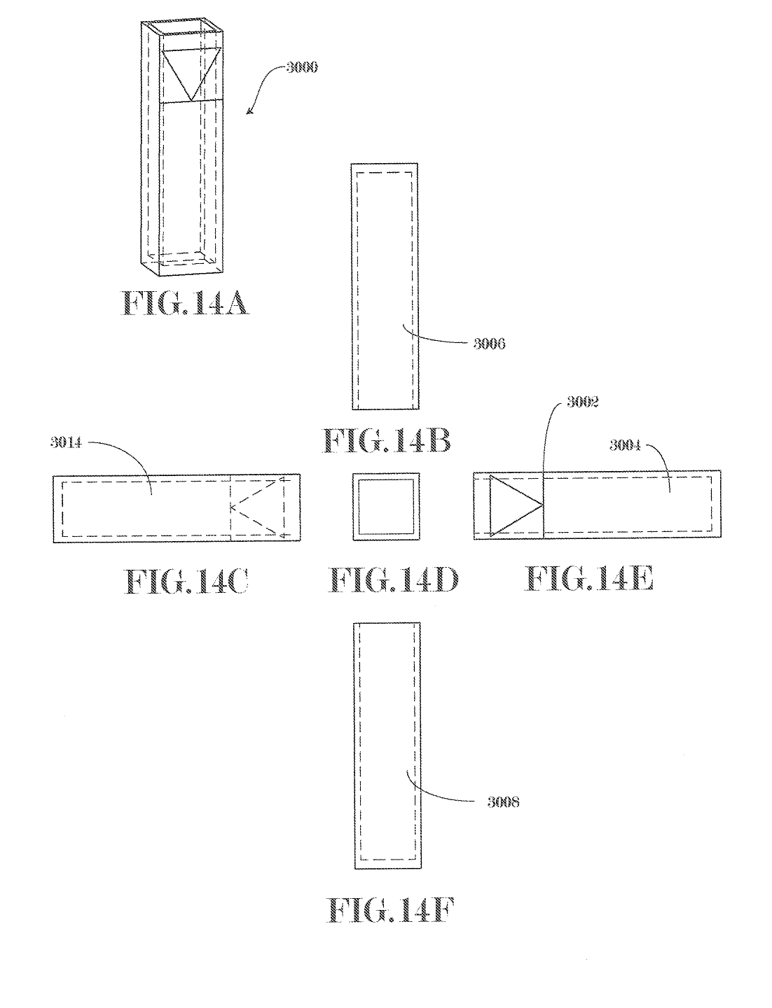

Cuvette cartridge and storage system see FIG. 16A, 3200 has a housing 3402 see FIG. 16C with 4 exterior walls with a smooth bottom, 2 interior walls 3424, 3420, a lid 3404, a seal 3408, an opening for gear 3426 and an opening for cuvettes to exit FIG. 16A,3202. Gear see FIG. 16C, 3502 protrudes from cuvette cartridge 3200 wall in a manner that a operator can remove the cuvette cartridge 3200 and replace with a new cartridge full of clean cuvettes see FIG. 14A, 3000 and gears 3502,1412 mesh on contact. Holes see FIG. 16C, 3416 placed on the front of the cartridge allow for easy removal of the cuvette cartridge. A shipping bar 3406 with a finger hole 3430 for easy removal is used to secure cuvettes as by going into slots 3418 and 3422 depicted in FIG. 16D. The shipping bar is removed after the cuvette cartridge is placed in testing unit in cuvette slot FIG. 5, 1006 but before the unit is ready for testing. The tape is attached to the cuvettes see FIG. 14A,3000 in such a way that it does not touch surfaces 3014 or 3004 as this would lead to possible incorrect readings by optical sensor. Tape couples the cuvettes above line 3002 to not interfere with optical sensor or light sources. Cuvettes are coupled together by a tape FIGS. 16B,16D,16E, 3102. Tape is attached to the cuvettes and is fed through a pin 3412 and is attached to a spool 3506. Pin 3412 is supported by bearings 3414, 3415.

Switch see FIG. 6B, 1504 is used to detect that the cuvette cartridge is in the correct place. Pegs FIG. 6A,1406 are used to guide the cuvette cartridge in place.

When gear 3502 is turned the spool 3506 which is attached to the gears with screws see FIG. 16E, 3508. As tape is taken up on the spool the tape pulls cuvettes forward until it reaches point FIG. 16D, 3302. At point 3302 the tape 3102 is dismounted from the cuvette. The cuvette is then pushed forward by the following cuvette. The cuvettes continue to advance until the optical sensor FIG. 6B,1714 detects a cuvette. Three openings FIG. 13C, 1709,1710,1711 allow for a clear view until a cuvette is present.

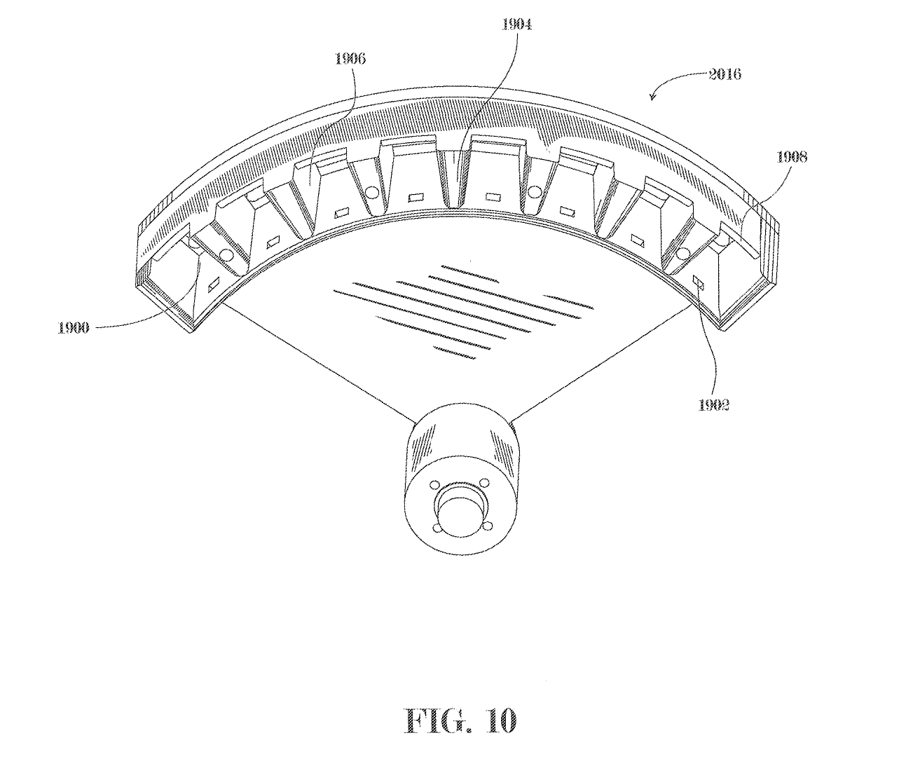

FIG. 6A shows the cuvette rotor 2016 located under water sample receiver 2100 and under reagent rotor 1002. The cuvette rotor has a plurality of cuvette slots. FIG. 10, shows the cuvette rotor 1900 an preferred embodiment, with cuvette slot slots divided by a wall 1904, each cuvette slot has a window 1908 for receiving light from the light source FIG. 6A,1440, a window 1902 for letting light pass through the sample to the light sensor FIG. 6A,1620. The walls 1906 of each cuvette slots is tapered to allow for some miss alignment. The sides of the cuvette slots are such that they fit loosely around the cuvette, this will allow proper mixing by letting the wheel move clockwise then counterclockwise or vice versa without making immediate contact on the returning motion.

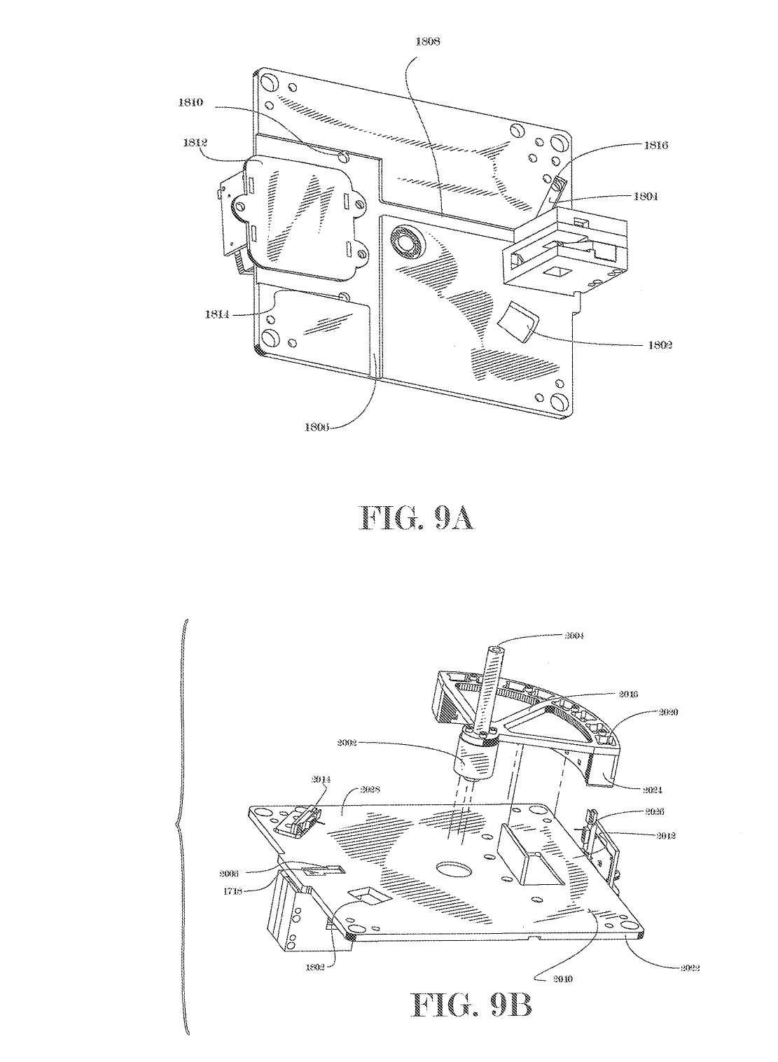

Once the cuvette is loaded into the cuvette loader FIG. 13B, 1700 a stepper motor FIG. 6A, 1506 moves shaft FIG. 9B, 2002 that is attached to the cuvette rotor 2016 in a way that position 2006 on the slide plate 2022 and the first cuvette bay 2020 are aligned. Alignment is assured by using sensor 2026 and looking for the leading edge of cuvette wheel surface 2024. This resets a rotary encoder FIG. 6A,1212 to keep track of the real-time position of the cuvette rotor 2016.

The cuvette cartridge 3200 moves cuvettes one by one into the cuvette loader 1700 which communicates with the controller and moves the cuvette rotor to the correct slot. Each time a cuvette has been loaded sensor FIG. 6B, 1714 verifies the actions have loaded a cuvette into the correct cuvette bay by moving the cuvette slot in front of the sensor FIG. 6A, 1502. Then each cuvette is then moved to spot 2028 where it is aligned with the users water in the water sample container receiver 2100. The linear stepper move down until the touch sensor 2404 makes contact with the water sample container. The sample container FIG. 12B, 2112 then releases water through a hole 2134 in the bottom of users water sample container. An optical sensor FIG. 12A, 2110 measures the drops similar to EP19870301700 by Spani. The optical sensor commutates with a controller 1202 to control the stepper motor 2416 depending on the test performed depends on the amount of drops of user sample water needed. This information is processed by controller 1202. After water has been added the cuvette is moved to a light source 1440 and optical receiver 1620. The baseline color of the individual's water is assessed. The cuvette is then moved to the reagent receiving area 2010 under reagent rotor.

The slide plate FIG. 6A, 2022 is a surface made of a industrial slide plastic such as Delrin® which features such a wire channels FIG. 19A, 1804, 1806,1808 to keep wires organized, holes 1810,1814,1816 for wires move out of plate 2022 and a place to mount the optical sensor 2014, optical receiver 1620 and light source 1440 in a way that keeps the top surface smooth, a waste shoot 1802 and a place to have cuvettes raise through the floor with a cuvette lifter FIG. 9B,1718.

The reagent rotor consist of a top FIG. 6A, 1206 with slots for reagents FIG. 5, 803 with numbers 804, a set at least 4 stand offs FIG. 6A, 1018 to space the plates apart correctly, a bottom plate 1442 which has a zero indicator FIG. 6A,1020, a channel FIG. 6A, 1208 for bearings FIG. 6B, 1404 to support the bottom plate, and a gear FIG. 6B 1424. Gear 1418 is connected to a motor and turns gear 1424 mounted to the reagent rotor FIG. 6A, 1002 and gear FIG. 6B, 1418 mesh together. The wheel is positioned by the controller using the rotary encoder FIG. 6A, 1210 and the feature 1020 passing through optical sensor FIG. 6B, 1624.

The reagent rotor keeps current inventory by using a barcode scanner FIG. 6A, 1010 and barcoded reagent cartridge 2800 which passes barcode information to a controller to keep inventory. Reagent inventory is maintained in a database. This database keeps expiration, lot numbers and installation date as well as other information. The level of each reagent is also maintained and periodically checked for accuracy using the same touch method used for the users sample water.

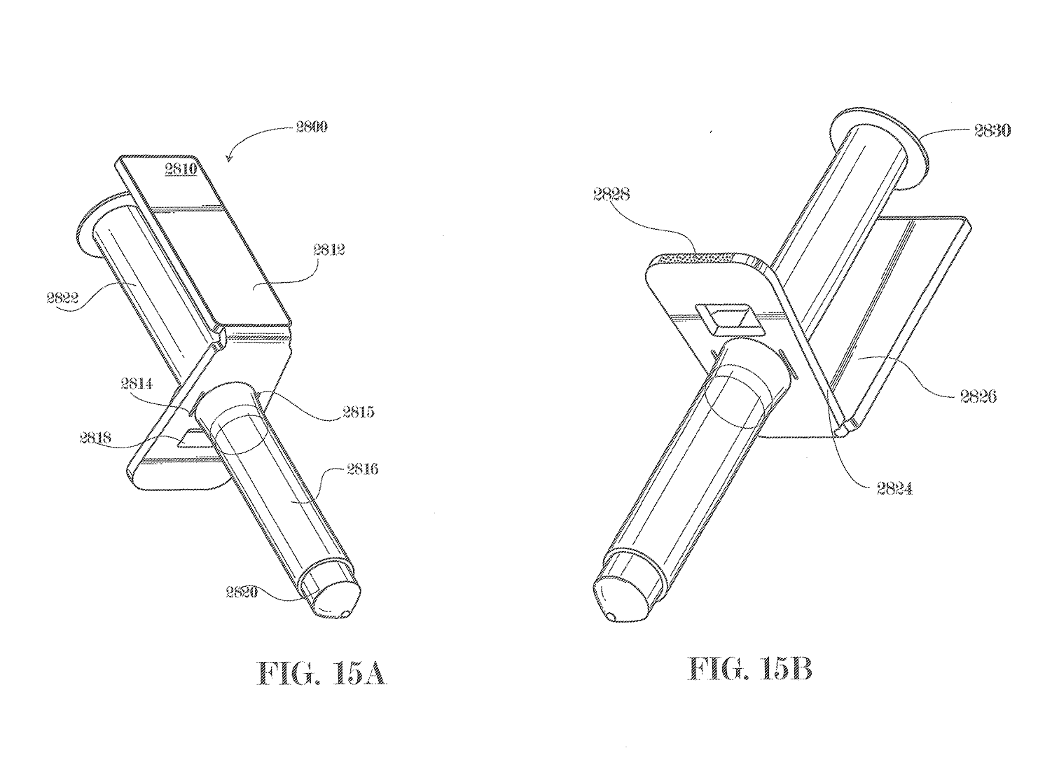

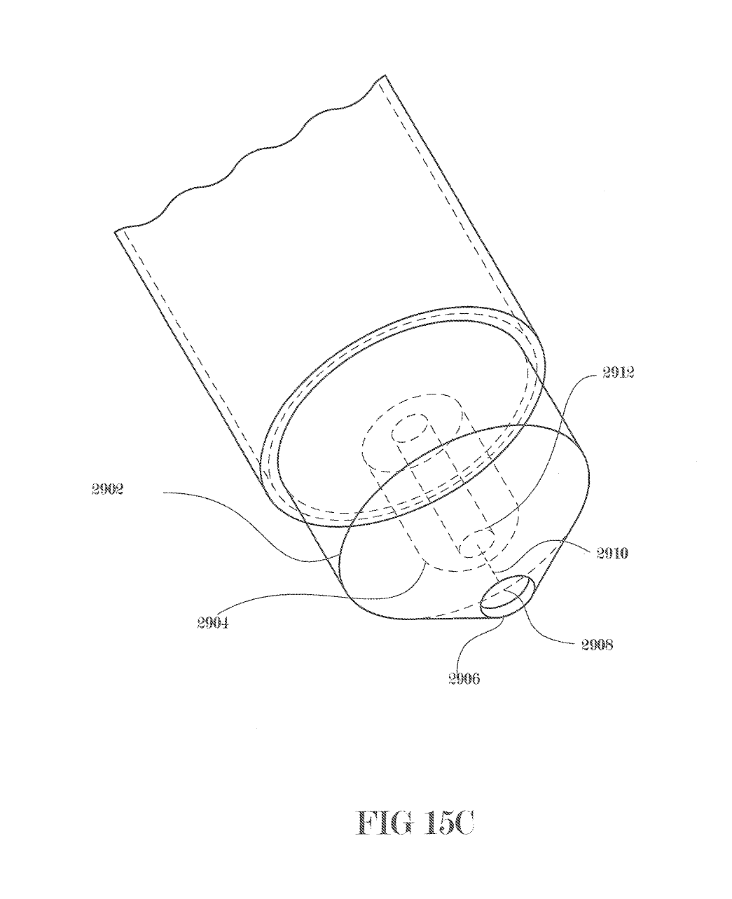

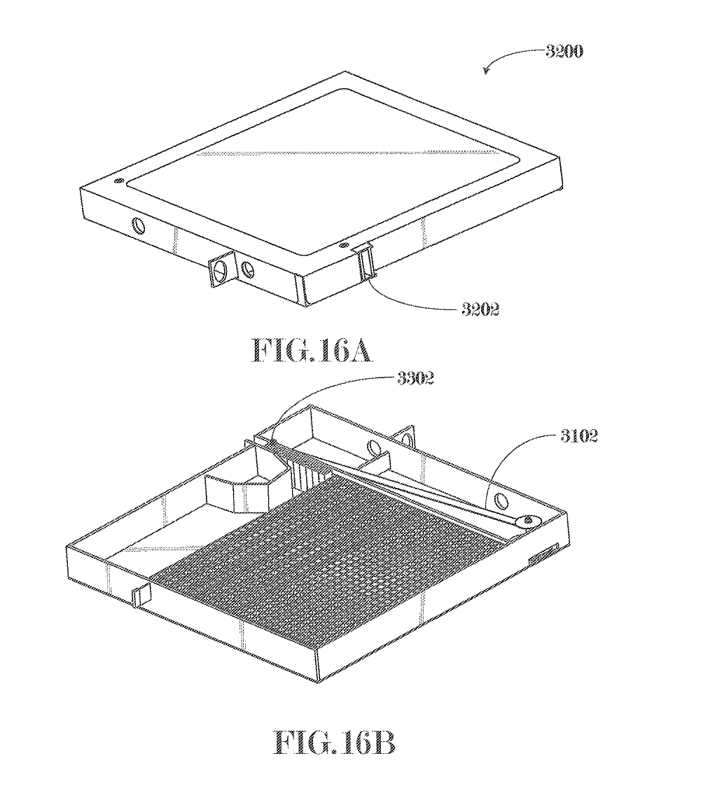

The one embodiment of reagent cartridge FIG. 15A 2800 consists of a housing 2812 to hold a barcode 2810 and protect syringe depressor 2822 from being depressed accidently, a syringe barrel 2816 with a self-sealing tip 2820, a depressor 2822 with a large surface 2830, a set of glue slots 2814, 2815 to hold syringe barrel 2816 in housing 2812, a self sealing tip 2820, a window 2818 for viewing a reagent wheel numbers 804, written info about what reagent is contained in the package in area 2828 and is color coded so that reagents types are easily understood. The surface behind the depressor 2826 has a graphical element to indicate the current reagent level. Surface 2824 is angled at such a way that when a series of reagent cartridge are placed next to each other they are self locking.

FIG. 15C shows the self sealing syringe tip comprised of a conical opening 2906, a syringe receiver 2904, a chamfered edge 2902, a pin hole channel made after rubber is molded 2910, and a drop point 2906. The seal does not allow air to react with reagent when being stored in the kiosk. The conical shape of feature 2908 allows the drop to fall out of the center of the feature 2908. This tip slips over an oral type syringe.

Reagent depressor 2300 is placed above spot FIG. 9B, 2010 on the slide plate 2022 so that a cuvette FIG. 14A will/can be placed under the depressor 2300 by the cuvette rotor 2016 and a reagent cartridge 2800 can be placed in between depressor and cuvette.

When a cuvette is placed in position 2010 and the needed reagent is located over top of the cuvette 2304, reagent extractor 2300 is moved down by motor FIG. 6B 1618 until sensor 2404 on reagent extractor 2300 is touched by surface FIG. 15B, 2830 on reagent cartridge FIG. 15A, 2800. The motor changes speeds to slowly extract reagent from reagent cartridge 2800. An optical sensor FIG. 6B, 1626 counts drops, Spani EP19870301700, as they fall into cuvette FIG. 16A.

The cuvette rotor FIG. 6A, 2016 will then move clockwise then counterclockwise in short burst to mix reagents and sample water. The cuvette rotor 2016 will then move the cuvette back to the optical sensor FIG. 6B, 1620 and light source FIG. 6A, 1440 where a reading will be taken. This process may require more than one reagent. After reading is taken, more reagents may need to be added in a titration testing style like patent U.S. Pat. No. 8,722,413 B1.

Another test type such as iodine may be time based. The controller would start a timer function timing a reaction when the reagent and water sample mix and wait for a reaction to occur. After a given amount of time the control system would receive a command to get an optical reading.

Optical reading would be processed by the controller and return a numeric value of concentration. The cuvette FIG. 16A would then travel to the waste bin 908 filled with an absorbent by moving the cuvette rotor to position FIG. 11B, 1802 on the slide tray where a hole is located to allow cuvettes to fall in to said bin.

Another water testing implement contemplated is specifically configured according to a particular implementation as a testing kiosk that uses test strips, U.S. Pat. No. 4,904,605 A, to test water. Where tests strips make contact with the water after the user has placed water in a water sample receiver to test water and give advice. The water from a user would be placed in a holder in a way that a test strip could come into contact with a water sample. An optical sensor would then be used to observe changes in said strip.

Another water testing implement contemplated is specifically configured according to a particular implementation as a testing kiosk where a continuous test strip or reel, WO2006003657 A3, is adapted in a way that the user places the water into the users water container receiver and a test strip is automatically contacted with the users water.

Another water testing implement contemplated specifically is configured according to a particular implementation as a testing kiosk that uses test strips, U.S. Pat. No. 4,904,605 A, to test water. Where tests strips make contact with the sample water by the user dipping the test strip and the user placing a reacted strip into a test strip reader, U.S. Pat. No. 8,703,057 B2.

It is contemplated that the automation of moving test strips inside may not be cost effective, in this case a user would react a test strip out side of the kiosk then place the strip in a shuttle for reading.

This embodiment testing implement contemplated is of a water testing implement specifically configured according to a particular implementation as a testing kiosk that uses probes, U.S. Pat. No. 6,395,158, to test water. This embodiment uses testing probes to test water. Probes are either placed in sample by user or by a mechanism that allows water to come in contact with probes.

When a customer/individual approaches a public use kiosk 2 (FIG. 1) to test water, the customer follows the instructions on the graphical interface 6. The customer is preferably given the option to select the test(s) to perform or login to a previously created account. Either through choosing an observed issue, a suggestion based on past test, a preselected bundle in the account setting, or choosing individual test(s).

A public use kiosk is preferably connected to the internet in a manner information can be exchanged with remote database(s) to allow users to access past results, create accounts, add data, share data and receive reminders through a website or other digital means. It also allows previously stored information from one kiosk to be shared with another kiosk and manage a profile online. Payments are preferably processed or verified over the connection as well.

Graphical interface 6 is preferably used to guide the customer in how to put the water in the water sample receiver 2100. The user would be instructed to close the water sample receiver in contemplated embodiment where water sample receiver is closable. Implement completes analysis and returns results of analysis to controller. The controller saves this data referenced with random characters and a randomly generated password. Said characters are given to the user via result printout, so the customer can later add tested data to there profile or create a new profile with data.

Embodiments contemplated above may have test chosen based on implements ability. A test strip reader would preferably test all pads on said strip or roll depending on the configuration.

Through a scanner, such as a bar code scanner 220 mounted somewhere on the kiosk 2. The bar code scanner 220, for example, may be configured to recognize a bar code on the water sample container, take payment, scan a loyalty card, or place information in users profile.

It will be understood that implementations are not limited to the specific components disclosed herein, as virtually any components consistent with the intended operation of a method and/or system implementation for a water testing kiosk may be utilized. Accordingly, for example, although particular water testing kiosk implementation components may be disclosed, such system components may comprise any shape, size, style, type, model, version, class, grade, measurement, concentration, material, weight, quantity, and/or the like consistent with the intended operation of a method and/or system implementation for a public water testing kiosk.

The invention claimed is:

1. A public use water testing kiosk comprising:
A housing;
A water sample receiver outside of the housing and accessible to a user from outside the housing;
A testing implement;
A graphical user interface coupled to the housing and responsive to the controller;
A printer;

2. The public use water testing kiosk of claim 1, further comprising:
A motorized rotor with multiple reagent storage systems that can move said reagent systems to cuvettes;
A fixture for removing water from sample container;
A trash receptacle for used consumables;
A water reservoir for spilled or discarded water;
A fixture for removing reagent from the reagent storage;
A light source and optical sensor placed so that light can travel through the cuvette while in cuvette rotor;
An optical system for measuring liquids dispensed from reagent systems;
A cuvette storage and dispensing system;
A lifter that moves a cuvette from storage system;
A cuvette rotor with multiple slots for cuvettes to be placed which moves cuvettes to needed location, on a circular path, moves back and forth motion to mix liquids, and can discard cuvette when finished

3. The public use water testing kiosk of claim 1, further comprising of the testing implement using test strips.

4. The public use water testing kiosk of claim 1, further comprising of the testing implement using test reels.

5. The public use water testing kiosk of claim 1, further comprising of a set of probes to analyze water.

6. A method of testing water at a public use water testing kiosk, the method comprising:
interacting with a graphical user interface; receiving water from the user;
displaying the results on the graphical user interface and issuing a printed report to the user.

7. A method of claim 6, wherein;
receiving a water sample from a user outside the kiosk through a water receiver on a kiosk housing, the kiosk housing enclosing water testing device such that the user cannot access a test equipment from outside the housing when interacting with the kiosk;
The sample water is dispensed into at least one cuvette;
An optical measurement of the water is taken;
At least one reagent is added to cuvette containing sample water;
The reagents and sample water is mixed with the cuvette rotor motion;
A second optical measurement is taken of the water;
The cuvette is discarded;

8. A method of claim 6, wherein;
Sample water makes contact with a test strip; A test strip is retrieved from a user;
A optical device views the strip; Test strip is discarded;

9. A method of claim 6, wherein;
A user interacts with a graphical user interface,
A new test strip is retrieved from a automatic test strip storage system;
Sample water makes contact with a test strip; A optical device views the strip;
Test strip is discarded;

10. A method of claim 6, wherein;
A user interacts with a graphical user interface,
A new test strip is retrieved from a automatic test reel storage system;
Sample water makes contact with a test strip;
A optical device views the strip;
Test strip is discarded;

11. A method generating water care recommendations, the method comprising: storing the data related to the current test results;
Retrieving individual's previous data;
Analyzing the data from the current test results; Analyzing the data from any past results; Issuing a printout containing advice;

12. A cuvette cartridge and storage system comprised of;
an opening for cuvettes to exit;
tape system;
not requiring the movement of all the cuvettes in the cartridge;
powered by an outside source;
not contain ribs or channels.
